SEQUENCE LISTING (43 sequences)

SEQ ID NO: 1 - RNA, 61 nt, Homo sapiens
cguguucacagcggaccuugauuuaaauguccauacaauuaaggcacgcggugaaugccaa

SEQ ID NO: 2 - RNA, 22 nt, Homo sapiens
cguguucacagcggaccuugau

SEQ ID NO: 3 - RNA, 22 nt, Homo sapiens
uaaggcacgcggugaaugccaa

SEQ ID NO: 4 - RNA, 58 nt, Homo sapiens
accgauuucuccugguguucagagucuguuuuugucuagcaccauuugaaaucgguua

SEQ ID NO: 5 - RNA, 23 nt, Homo sapiens
accgauuucuccugguguucaga

SEQ ID NO: 6 - RNA, 22 nt, Homo sapiens
uagcaccauuugaaaucgguua

SEQ ID NO: 7 - RNA, 62 nt, Homo sapiens
agaucagaaggugauuguggcuuuggguggauauuaaucagccacagcacugccuggucaga

SEQ ID NO: 8 - RNA, 22 nt, Homo sapiens
agaucagaaggugauuguggcu

SEQ ID NO: 9 - RNA, 21 nt, Homo sapiens
ccacagcacugccuggucaga

SEQ ID NO: 10 - RNA, 65 nt, Homo sapiens
uggcagugucuuagcugguuguugugagcaauaguaaggaagcaaucagcaaguauacugcccua

SEQ ID NO: 11 - RNA, 22 nt, Homo sapiens
uggcagugucuuagcugguugu

SEQ ID NO: 12 - RNA, 22 nt, Homo sapiens
aaucagcaaguauacugcccua

SEQ ID NO: 13 - RNA, 60 nt, Homo sapiens
uguaaacauccuacacucagcuguaauacauggauuggcugggagguggauguuuacuuc

SEQ ID NO: 14 - RNA, 22 nt, Homo sapiens
uguaaacauccuacacucagcu

SEQ ID NO: 15 - RNA, 22 nt, Homo sapiens
cugggagguggauguuuacuuc

SEQ ID NO: 16 - RNA, 61 nt, Homo sapiens
acucaaaaugggggcgcuuuccuuuuugucuguacugggaagugcuucgauuuuggggugu

SEQ ID NO: 17 - RNA, 22 nt, Homo sapiens
acucaaaaugggggcgcuuucc

SEQ ID NO: 18 - RNA, 23 nt, Homo sapiens
gaagugcuucgauuuuggggugu

SEQ ID NO: 19 - RNA, 84 nt, Homo sapiens
auguuggugcaaaaguaauuguggauuuugcuauuacuuguauuuauuuguaaugcaaaacccgcaauuaguuuugcaccaacc

SEQ ID NO: 20 - RNA, 22 nt, Homo sapiens
aaaaguaauuguggauuuugcu

SEQ ID NO: 21 - RNA, 96 nt, Homo sapiens
gcuccaguaacaucuuaaaguaaauaugcaccaaaauuacuuuugguaaauacaguuuuggugcauauuuacuuuaggauguuacuggagcuccca

SEQ ID NO: 22 - RNA, 21 nt, Homo sapiens
uaaaguaaauaugcaccaaaa

SEQ ID NO: 23 - RNA, 91 nt, Homo sapiens
aaacacuugagcccagcgguuugaggcuacagugagaugugauccugccacaucucacuguagccucgaaccccugggcucaagugauuca

SEQ ID NO: 24 - RNA, 22 nt, Homo sapiens
uuugaggcuacagugagaugug

SEQ ID NO: 25 - RNA, 22 nt, Homo sapiens
ucucacuguagccucgaacccc

SEQ ID NO: 26 - RNA, 85 nt, Homo sapiens
cucaggcugugacacucuagagggaagcgcuuucuguugucugaaagaaaggaaagugcauccuuuuagaguguuacuguuugag

SEQ ID NO: 27 - RNA, 22 nt, Homo sapiens
cucuagagggaagcgcuuucug

SEQ ID NO: 28 - RNA, 22 nt, Homo sapiens
aaagugcauccuuuuagagugu

SEQ ID NO: 29 - RNA, 60 nt, Homo sapiens
agagguugcccuuggugaauucgcuuuauuuauguugaaucacacaaaggcaacuuuugu

SEQ ID NO: 30 - RNA, 22 nt, Homo sapiens
agagguugcccuuggugaauuc

SEQ ID NO: 31 - RNA, 22 nt, Homo sapiens
aucacacaaaggcaacuuuugu

SEQ ID NO: 32 - RNA, 60 nt, Homo sapiens
cuccugacuccagguccuguguguuaccuagaaauagcacuggacuuggagucagaaggc

SEQ ID NO: 33 - RNA, 22 nt, Homo sapiens
cuccugacuccagguccugugu

SEQ ID NO: 34 - RNA, 22 nt, Homo sapiens
acuggacuuggagucagaaggc

SEQ ID NO: 35 - RNA, 90 nt, Homo sapiens
ccgggccccugugagcaucuuaccggacagugcuggauuucccagcuugacucuaacacugucugguaacgauguucaaaggugacccgc

SEQ ID NO: 36 - RNA, 22 nt, Homo sapiens
caucuuaccggacagugcugga

SEQ ID NO: 37 - RNA, 22 nt, Homo sapiens
uaacacugucugguaacgaugu

SEQ ID NO: 38 - RNA, 59 nt, Homo sapiens
cagcagcaauucauguuuugaaguguucuaaaugguucaaaacgugaggcgcugcuaua

SEQ ID NO: 39 - RNA, 22 nt, Homo sapiens
cagcagcaauucauguuuugaa

SEQ ID NO: 40 - RNA, 22 nt, Homo sapiens
caaaacgugaggcgcugcuaua

SEQ ID NO: 41 - RNA, 97 nt, Homo sapiens
cuagauaaguuauuaggugggugcaaagguaauugcaguuuuucccauuauuuuaauugcgaaaacagcaauuaccuuugcaccaaccugauggagu

SEQ ID NO: 42 - RNA, 22 nt, Homo sapiens
aaagguaauugcaguuuuuccc

SEQ ID NO: 43 - RNA, 22 nt, Homo sapiens
cgaaaacagcaauuaccuuugc

(19) United States
(12) Patent Application Publication
(10) Pub. No.: US 2021/0260141 A1
(43) Pub. Date: Aug. 26, 2021

(54) MSC- AND EXOSOME-BASED IMMUNOTHERAPY

(71) Applicant: EXOSTEM BIOTEC LTD., Tel Aviv (IL)

(72) Inventors: Aharon BRODIE, Meitar (IL); Chaya BRODIE, Southfield, MI (US)

(21) Appl. No.: 17/261,078

(22) PCT Filed: Jul. 18, 2019
(86) PCT No.: PCT/IL2019/050813

§ 371 Date: Jan. 18, 2021

Related U.S. Application Data
(60) Provisional application No. 62/699,845, filed on Jul. 18, 2018.

(51) Int. Cl. A61K 35/768; A61K 35/28; A61K 35/50; A61K 31/7105; A61K 45/06; A61K 38/17

(52) U.S. Cl. CPC A61K 35/768; A61K 35/28; A61K 38/1774; A61K 31/7105; A61K 45/06; A61K 35/50

(57) ABSTRACT

Compositions comprising at least one of miR that down regulates PD-L1/PD-1, MSCs expressing same, or exosomes from those MSCs are provided. Methods of decreasing PD-L1 expression, PD-1 expression or both and treating PD-L1 positive cancers using same are also provided.

### CROSS REFERENCE TO RELATED APPLICATIONS

This application claims the benefit of priority of U.S. Provisional Patent Application No. 62/699,845, filed Jul. 18, 2018 entitled “MSC-BASED IMMUNOTHERAPY”, the contents of which are incorporated herein by reference in their entirety.

### FIELD OF INVENTION

The present invention is in the field of PD-L1/PD-1 cancer therapy.

### BACKGROUND OF THE INVENTION

Anti-programmed death protein 1 (PD-1) or anti-PD ligand 1 (PD-L1) antibody treatments cause long-lasting antitumor responses in patients with many types of cancer. In fact, these treatments have become a standard of care treatment for patients with various tumors such as metastatic melanoma, carcinomas of the head and neck, lung, kidney, and bladder, Merkel cell carcinoma, and Hodgkin disease. These antibodies function by blocking the PD-L1/PD-1 axis that some cancers use to inhibit an anti-tumor immune response. When the axis is blocked T-cells are freed to activate and target the tumor cells.

Only a subset of patients responds to these therapies, whereas many patients exhibit resistance to inhibition of PD-1/PD-L1. Those that do respond also frequently relapse after a period of time. As such, new anti-PD-1/PD-L1 therapies, and methods of improving preexisting therapies, are greatly needed.

It has been reported that some non-responder patients lack CD8+T cells in the tumor sites. This lack of CD8+T cells within a tumor renders the PD-1/PD-L1 blockade therapy ineffective as the dearth of cells means an effective immune response cannot be mounted. One of the approaches to improve this therapy is to perform combination immunotherapy which will simultaneously attract CD8+T cells into the tumors while also blocking the PD-1/PD-L1 axis. This can be done by altering the immune-suppressive tumor microenvironment (TME). One such approach to do this is intra-tumoral administration of oncolytic viruses that attract immune cells and increase CD8+T cell infiltration. Combined therapies that alter the immunosuppressive TME, recruit CD8+T cells and blockade the PD-1/PD-L1 axis are greatly needed.

Recent studies demonstrated that many tumor cells can release a large amount of their PD-L1 on the surface of exosomes rather than present these molecules on the cell surface. Moreover, it was demonstrated that exosomal PD-L1 inhibits T cell function and is resistant to anti-PD-L1 therapy. As such, exosomal PD-L1 acts in an additive manner rather than a redundant one to the cellular ligand. Thus, there is an urgent need to develop therapies that can target the secreted PD-L1.

### SUMMARY OF THE INVENTION

The present invention provides compositions comprising at least one of miR-124, miR-29c, and miR-383, MSCs expressing same, or exosomes from those MSCs, as well as kits comprising same. Methods of decreasing PD-L1 expression, PD-1 expression or both and of treating PD-L1 positive cancers are also provided.

According to a first aspect, there is provided a method of decreasing Programmed Death Ligand 1 (PD-L1) expression, PD-1 expression, or both, in a cell, the method comprising contacting the cell with a mesenchymal stem cell (MSC), extracellular vesicle from the MSC or both.

According to some embodiments, the MSC is derived from umbilical cord (UC) or chorionic placenta (CH). According to some embodiments, the MSC is a CH-MSC.

According to some embodiments, the cell is a cancer cell.

According to another aspect, there is provided a method of decreasing proliferation of a cancer cell, the method comprising contacting the cancer cell with a chorionic placenta or umbilical cord MSC, extracellular vesicle from the MSC or both.

According to some embodiments, the cell is in a subject.

According to some embodiments, the decreasing proliferation comprises decreasing self-renewal of the cell and increasing immune surveillance against the cell. According to some embodiments, the decreasing comprises at least a 20% decrease.

According to some embodiments, the MSC, extracellular vesicle from the MSC or both, comprise at least one exogenous microRNA (miR) that binds to and inhibits expression of PD-L1, PD-1 or both. According to some embodiments, the at least one exogenous miR is not oncogenic. According to some embodiments, the at least one exogenous miR binds to a 3′ untranslated region (UTR) of PD-L1.

According to some embodiments, the at least one exogenous miR is selected from the group consisting of: miR-124, miR-29c, miR-383, miR-373, miR-548, miR-559, miR-1304, miR-519, miR-377, miR-378, miR-200, miR-424, miR-570, and miR-34.

According to some embodiments, the at least one exogenous miR is selected from the group consisting of: miR-373, miR-548, miR-559, miR-1304, miR-519, miR-377, miR-378, miR-200, miR-424, miR-570, and miR-34.

According to some embodiments, the at least one exogenous miR binds to a 3′ UTR of PD-1.

According to some embodiments, the at least one exogenous miR is selected from the group consisting of: miR-124, miR-34 and miR-30b.

According to some embodiments, the at least one exogenous miR is miR-124.

According to some embodiments, the MSC, exosomes from the MSC or both, comprise at least two exogenous miRs selected from the group consisting of: miR-124, miR-29c, miR-383, miR-34, miR-30b, miR-373, miR-548, miR-559, miR-1304, miR-519, miR-377, miR-378, miR-200, miR-424, and miR-570.

According to some embodiments, the MSC, exosomes from the MSC, or both comprises miR-124 and at least one miR selected from the group consisting of miR-29c, miR-383, miR-34, and miR-30b.

According to some embodiments, the MSC, exosomes from the MSC, or both comprise miR-124 and
a. miR-29c and miR-383; or
b. miR-34 and miR-30b.

According to some embodiments, the cancer cell is selected from a brain cancer cell, a lung cancer cell, a breast cancer cell, a melanoma cell, a meningioma cell, a pancreatic cancer cell, a prostate cancer cell, a medulloblastoma cell, a glioma cell and a metastatic cell of a brain cancer.

According to some embodiments, the cancer cell is a brain cancer cell. According to some embodiments, the brain cancer is glioblastoma multiform (GBM). According to some embodiments, the GBM cell is a GBM stem cell.

According to some embodiments, the MSC or exosomes expresses exogenous membranal TRAIL (mTRAIL) protein.

According to some embodiments, the MSC or exosomes is infected with an oncolytic virus.

According to some embodiments, the oncolytic virus is Newcastle disease virus (NDV).

According to another aspect, there is provided a pharmaceutical composition comprising at least one of:
a. an MSC expressing at least one exogenous miR selected from miR-124, miR-29c miR-383 miR-373, miR-548, miR-559, miR-1304, miR-519, miR-377, miR-378, miR-200, miR-424, miR-570, and miR-34;
b. extracellular vesicles derived from (a);
c. a combination thereof.

According to some embodiments, the MSC is selected from a UC MSC and a CH MSC.

According to some embodiments, the MSC is selected from a bone marrow MSC, adipose MSC and an amniotic placenta MSC and expressed exogenous miR-124.

According to some embodiments, the MSC further comprises at least one anti-cancer therapeutic agent.

According to some embodiments, the at least one anti-cancer therapeutic agent is selected from mTRAIL protein or FAS ligand (FasL).

According to some embodiments, the MSC expresses an oncolytic virus. According to some embodiments, the oncolytic virus is NDV.

According to some embodiments, the composition of the invention comprises at least two miRs selected from miR-124, miR-29c and miR-383.

According to some embodiments, the composition of the invention comprises miR-124, miR-29c and miR-383.

According to some embodiments, the MSC comprises at least two miRs selected from miR-124, miR-29c and miR-383.

According to some embodiments, the MSC comprises miR-124, miR-29c and miR-383.

According to some embodiments, the composition of the invention further comprises at least one exogenous miR selected from miR-34 and miR-30b.

According to some embodiments, the MSC comprises at least one exogenous miR selected from miR-34 and miR-30b.

According to some embodiments, the composition of the invention is for use in treating a PD-L1 positive cancer in a subject in need thereof.

According to another aspect, there is provided a pharmaceutical composition for use is treating a PD-L1 positive cancer in a subject in need thereof, wherein the pharmaceutical composition comprises at least one miR, or a synthetic oligonucleotide mimic of the at least one miR, selected from a group consisting of miR-124, miR-29c, miR-383, miR-373, miR-548, miR-559, miR-1304, miR-519, miR-377, miR-378, miR-200, miR-424, miR-570, and miR-34.

According to some embodiments, the at least one miR, or a synthetic oligonucleotide mimic of the at least one miR is within an exosome.

According to another aspect, there is provided a method of treating a Programmed Death Ligand 1 (PD-L1) positive cancer in a subject in need thereof, the method comprising administering to the subject a pharmaceutical composition of the invention.

According to some embodiments, the treating comprises decreasing PD-L1 expression by the PD-L1 positive cancer.

According to some embodiments, the treating comprises decreasing proliferation of the PD-L1 positive cancer.

According to some embodiments, the treating comprises decreasing PD-1 expression by the subject.

According to some embodiments, the treating comprises converting a cancer refractory to PD-L1/PD-1 based therapy to a cancer responsive to PD-L1/PD-1 based therapy.

According to some embodiments, the MSC is allogenic or autologous to the subject.

According to some embodiments, the decreasing comprises at least a 20% decrease.

According to some embodiments, the PD-L1 positive cancer is selected from a brain cancer cell, a lung cancer cell, a breast cancer cell, a melanoma cell, a meningioma cell, a pancreatic cancer cell, a prostate cancer cell, a medulloblastoma cell, a glioma cell and a metastatic cell of a brain cancer.

According to some embodiments, the PD-L1 positive cancer is a brain cancer. According to some embodiments, the brain cancer is glioblastoma multiform (GBM).

According to some embodiments, the MSC comprises an anti-cancer therapeutic.

According to some embodiments, the therapeutic is exogenous membranal TRAIL protein.

According to some embodiments, the therapeutic is Fas ligand (FasL).

According to some embodiments, the MSC is infect by an oncolytic virus.

According to some embodiments, the oncolytic virus is NDV.

According to some embodiments, the method of the invention further comprises administering at least one anticancer treatment.

According to some embodiments, the at least one anticancer treatment is selected from irradiating the subject and chemotherapy.

According to some embodiments, the MSCs, exosomes from the MSCs, or both sensitize the PD-L1 positive cancer to irradiation, chemotherapy or both.

According to some embodiments, the MSCs, exosomes from the MSCs or both protect healthy cells from the irradiation, chemotherapy or both.

According to some embodiments, the at least one anticancer treatment is a PD-L1/PD-1 therapeutic.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-B: Bar graphs of (1A) GSCs self-renewal after coculture with CH-, UC- and AD-MSCs as well as their exosomes expressing various miRs, (1B) relative PD-L1 luciferase reporter activity after coculture with CH- , UC- and AD-MSCs, expressing various miRs.

FIGS. 2A-B: Bar graphs of (2A) relative PD-1 luciferase reporter activity and (2B) self-renewal of GSCs after coculture with CH-MSCs or their exosomes.

FIGS. 3A-B: Bar graphs of relative PD-L1 luciferase reporter activity (3A) in various cancers after coculture with CH-MSCs expression miR-124 and (3B) in GSCs.

FIG. 4: Bar graph of relative self-renewal of 4 brain cancer stem cell lines.

FIG. 5: Bar graph of relative PD-L1 luciferase reporter activity after coculture of GSCs with exosomes from CH-MSCs expressing various miRs.

### DETAILED DESCRIPTION OF THE INVENTION

The present invention, in some embodiments, provides compositions for treat eating cancer, decreasing expression of PD-L1, decreasing expression of PD-1 and combinations thereof. Methods of so doing are also provided.

The present invention is based, at least in part, on the surprising finding that unmodified chorionic placenta (CH) and umbilical cord (UC) derived MSCs downregulate expression of programed death-ligand 1 (PD-L1) and its receptor (PD-1). Further, these unmodified MSC strongly downregulate cancer stem cell self-renewal. Unexpectedly, MSCs of other origins do neither. It was further found that 3 miRs, miR-124, miR-29c and miR-378 which had previously never been implicated in PD-L1 regulation strongly inhibit PD-L1 expression in cancer cells. Additional anti-PD-L1 miRs were also found. When the MSCs and miRs were combined a strong synergistic effect was observed with PD-L1 reporter activity dropping below 20% and self-renewal dropping below 30%.

It was further discovered that miR-124, miR-34 and miR-30b which had previously never been implicated in PD-1 regulation, strongly inhibit PD-1 expression. Thus miR-124 and mir-34 target both sides of the PD-L1/PD-1 axis. Combination of these miRs with MSCs also had a strong synergistic effect on both PD-1 reporter activity and self-renewal. Additionally, combinations of these MSCs, miRs and other cytotoxic agents had strong synergistic effects on various cancer stem cells (CSC), and strongly inhibited CSC self-renewal.

Finally, it was found that the silencing of PD-L1 by specific miRNAs decreases not only the cell-expressed PD-L1 but also the expression of PD-L1 on secreted exosomes. Importantly, this reduction in circulating PD-L1 promotes T cell activity and systemic anti-tumor immunity. The inhibition of exosomal PD-L1 expression also provides a method of treatment for to tumors resistant to known anti-PD-L1 antibodies.

By a first aspect, there is provided a pharmaceutical composition comprising at least one miR, pre-miR or a synthetic oligonucleotide mimic of at least one miR selected from miR-124, miR-29c and miR-383.

By another aspect, there is provided a pharmaceutical composition comprising at least one miR, pre-miR or a synthetic oligonucleotide mimic or the at least one miR selected from miR-124, miR-34 and miR-30b.

By another aspect, there is provided a pharmaceutical composition comprising at least one miR, pre-miR or a synthetic oligonucleotide mimic of the at least one miR selected from miR-124, miR-29c, miR-383, miR-34, miR-30b, miR-373, miR-548, miR-559, miR-1304, miR-519, miR-377, miR-378, miR-200, miR-424, and miR-570.

By another aspect, there is provided a pharmaceutical composition comprising an MSC over-expressing at least one miR, pre-miR, oligonucleotide mimic of a miR selected from miR-124, miR-29c, miR-383, miR-34 and miR-30b.

By another aspect, there is provided a pharmaceutical composition comprising extracellular vesicles from an MSC expressing at least one miR, pre-miR or s synthetic oligonucleotide mimic of the at least one miR selected from miR-124, miR-29c, miR-383, miR-34, miR-30b, miR-373, miR-548, miR-559, miR-1304, miR-519, miR-377, miR-378, miR-200, miR-424, and miR-570.

By another aspect, there is provided a pharmaceutical composition comprising at least one of:
a. at least one miR, pre-miR, or a synthetic oligonucleotide mimic of at least one miR, selected from miR-124, miR-29c, miR-383 miR-34, and miR-30b;
b. an MSC over-expressing at least one exogenous miR, pre-miR or s synthetic oligonucleotide mimic of the at least one miR selected from miR-124, miR-29c, miR-383, miR-34, miR-30b, miR-373, miR-548, miR-559, miR-1304, miR-519, miR-377, miR-378, miR-200, miR-424, and miR-570;
c. extracellular vesicles derived from b;
d. a combination thereof.

By another aspect, there is provided a method of decreasing PD-L1 expression, PD-1 expression, or both, in a cell the method comprising contacting the cell with an MSC, extracellular vesicle from an MSC, or both.

By another aspect, there is provided a method of decreasing PD-L1 expression, PD-1 expression, or both, in a cell the method comprising contacting the cell with a pharmaceutical composition of the invention.

By another aspect, there is provided a method of decreasing proliferation of a cancer cell, the method comprising contacting the cancer cell with an MSC, extracellular vesicles from the MSC or both.

By another aspect, there is provided a method of decreasing proliferation of a cancer cell, the method comprising contacting the cancer cell with a composition of the invention.

By another aspect, there is provided a method of treating cancer in a subject in need thereof, the method comprising administering to the subject a composition of the invention.

By another aspect, there is provided a method of converting a subject refractory to PD-L1/PD-1 based therapy to a responsive subj ect to PD-L1/PD-1 based therapy, the method comprising administering to the subject an MSC, extracellular vesicles from the MSC or both.

By another aspect, there is provided a method of converting a subject refractory to PD-L1/PD-1 based therapy to a responsive subject to PD-L1/PD-1 based therapy, the method comprising administering to the subject a composition of the invention.

As used herein, the term “mesenchymal stem cell” or “MSC”, refers to multipotent stromal stem cells that have the ability to differentiate into osteoblasts, adipocytes, myocytes, chondroblasts, skeletal muscle cells and endothelial cells. MSC are present in the bone marrow, adipose tissue, peripheral blood, chorionic placenta, amniotic placenta, umbilical cord blood, and dental pulp, among other tissues. The term “multipotent” refers to stem cells which are capable of giving rise to many cell types. In some embodiments, the MSC is derived from umbilical cord (UC) or chorionic placenta (CH). In some embodiments, the MSC is derived from adipose, bone marrow, dental pulp, umbilical cord amniotic placenta or chorionic placenta. In some embodiments, the MSC is derived from adipose, bone marrow, dental pulp, umbilical cord or chorionic placenta. In some embodiments, the MSC is derived from dental pulp, umbilical cord or chorionic placenta. In some embodiments, the MSC is derived from chorionic placenta. In some embodiments, the MSC is derived from umbilical cord. In some embodiments, the MSC is derived from dental pulp. In some embodiments, the MSC is derived from any one of umbilical cord and chorionic placenta. In some embodiments, the MSC is not derived from amniotic placenta. In some embodiments, the pharmaceutical composition is devoid of amniotic placenta MSCs. In some embodiments, the pharmaceutical composition is substantially devoid of amniotic placenta MSCs. In some embodiments, the MSC is not a bone marrow derived MSC. In some embodiments, the MSC is not an adipose derived MSC. In some embodiments, the MSC is an unmodified MSC. In some embodiments, the MSC has been modified to over-express at least one miR of the invention. In some embodiments, the MSC is a bone marrow or adipose MSC and over expressed exogenous miR-124. In some embodiments, the MSC is an amniotic placenta, bone marrow or adipose MSC and over expressed exogenous miR-124.

In some embodiments, the MSC and/or its exosomes are allogenic to the subject. In some embodiments, the MSC and/or its exosomes are autologous to the subject. In some embodiments, all CH or UC MSCs are allogeneic to any subject. In some embodiments, the MSC and/or its exosomes are allogenic or autologous to the subject. In some embodiments, the MSC and/or its exosomes do not induce an immune response in the subject. MSC and especially their exosomes and extracellular vesicles have a strong advantage as a therapeutic as they do not express MHCII molecules and do not induce an immune response. Further MSCs and their exosomes actively inhibit the immune response. CH and UC MSCs and their exosomes are particularly effective in this respect. In this way the MSCs and/or their exosomes can be used as an “off the shelf” therapeutic agent that can be administered to any subject in need thereof.

Chorionic, and umbilical cord MSCs are well known in the art. In some embodiments, these MSCs or their secreted vesicles can be identified by examining the expression of various proteins, and regulatory RNA such as are described in international patent application WO/2018083700, the content of which are herein incorporated by reference in their entirety. In some embodiments, the MSCs are identified by the tissue they were isolated from.

In some embodiments, the composition comprises miR-124. In some embodiments, the MSC over-expresses miR-124. In some embodiments, the composition comprises miR-29c. In some embodiments, the MSC over-expresses miR-29c. In some embodiments, the composition comprises miR-383. In some embodiments, the MSC over-expresses miR-383. In some embodiments, the composition comprises miR-34. In some embodiments, the MSC over-expresses miR-34. In some embodiments, the composition comprises miR-30b. In some embodiments, the MSC over-expresses miR-30b. In some embodiments, the composition comprises miR-373. In some embodiments, the MSC over-expresses miR-373. In some embodiments, the composition comprises miR-548. In some embodiments, the MSC over-expresses miR-548. In some embodiments, the composition comprises miR-559. In some embodiments, the MSC over-expresses miR-559. In some embodiments, the composition comprises miR-1304. In some embodiments, the MSC over-expresses miR-1304. In some embodiments, the composition comprises miR-519. In some embodiments, the MSC over-expresses miR-519. In some embodiments, the composition comprises miR-377. In some embodiments, the MSC over-expresses miR-377. In some embodiments, the composition comprises miR-200. In some embodiments, the MSC over-expresses miR-200. In some embodiments, the composition comprises miR-424. In some embodiments, the MSC over-expresses miR-424. In some embodiments, the composition comprises miR-570. In some embodiments, the MSC over-expresses miR-570. In some embodiments, the composition comprises miR-124 and at least one other miR selected from miR-29c, miR-383 miR-34, miR-30b, miR-373, miR-548, miR-559, miR-1304, miR-519, miR-377, miR-378, miR-200, miR-424, and miR-570. In some embodiments, the MSC over-expresses miR-124and at least one other miR selected from miR-29c, miR-383 miR-34, miR-30b, miR-373, miR-548, miR-559, miR-1304, miR-519, miR-377, miR-378, miR-200, miR-424, and miR-570. In some embodiments, the composition comprises a plurality of miRs selected from miR-124, miR-29c, miR-383 miR-34, miR-30b, miR-373, miR-548, miR-559, miR-1304, miR-519, miR-377, miR-378, miR-200, miR-424, and miR-570. In some embodiments, the MSC over-expresses a plurality of miRs selected from miR-124, miR-29c, miR-383 miR-34, miR-30b, miR-373, miR-548, miR-559, miR-1304, miR-519, miR-377, miR-378, miR-200, miR-424, and miR-570. In some embodiments, the composition comprises miR-124, miR-29c, and miR-383. In some embodiments, the MSC over-expresses miR-124, miR-29c, and miR-383. In some embodiments, the composition comprises miR-124, miR-34, and miR-30b. In some embodiments, the MSC over-expresses miR-124, miR-34, and miR-30b. In some embodiments, the composition or MSCs comprise only these exogenous miRs and no others. It will be understood by a skilled artisan that any combination of these 5 miRs is possible, up to and include all 5 of them together, either in the composition or exogenously expressed in the MSC. In some embodiments, the MSC over-expresses 1, 2, 3, 4, 5, 6, 7, 8, 9, 10, 11, 12, 13, 14, or 15 miRs selected from miR-124, miR-29c, miR-383 miR-34, miR-30b, miR-373, miR-548, miR-559, miR-1304, miR-519, miR-377, miR-378, miR-200, miR-424, and miR-570. Each possibility represents a separate embodiment of the invention. In some embodiments, the miR is an exogenous miR.

Sequences of miRs and their pre-miRs can be found on many websites including, but not limited to, MirGeneDB, RNAcentral, and miRBase. In some embodiments, the sequences of the miRs of the invention and their pre-miRs are those found in Table 1. In some embodiments, the miR comprises the sequence found in Table 1. In some embodiments, the miR consists of the sequence found in Table 1. In some embodiments, the pre-miR comprises the sequence found in Table 1. In some embodiments, the pre-miR consists of the sequence found in Table 1. In some embodiments, the sequence of the pre-miR is that found in Table 1 without the 30 bp flank. In some embodiments, the pre-miR sequence for miR-548, miR-559, miR-1304, miR-519, miR-200 and miR-570 as provided in Table 1 comprises the 30 bp flank.

TABLE 1
Sequences

| | | mature mir | mature mir |
|---|---|---|---|
| | | sequence 5p | sequence 3p |
| mir | Pre-mir sequence (SEQ ID NO:) | (SEQ ID NO:) | (SEQ ID NO:) |
| mir- | cguguucacagcggaccuugauuuaaauguccaua | cguguucacagcgga | uaaggcacgcgguga |
| 124 | caauuaaggcacgcggugaaugccaa (1) | ccuugau (2) | augccaa (3) |
| miR- | accgauuucuccugguguucagagucuguuuuug | accgauuucuccugg | uagcaccauuugaaa |
| 29c | ucuagcaccauuugaaaucgguua (4) | uguucaga (5) | ucgguua (6) |
| miR- | agaucagaaggugauuguggcuuuggguggauau | agaucagaaggugau | ccacagcacugccug |
| 383 | uaaucagccacagcacugccuggucaga (7) | uguggcu (8) | gucaga (9) |
| miR- | uggcagugucuuagcugguuguugugagcaauag | uggcagugucuuagc | aaucagcaaguauac |
| 34 | uaaggaagcaaucagcaaguauacugcccua (10) | ugguugu (11) | ugcccua (12) |
| miR- | uguaaacauccuacacucagcuguaauacauggau | uguaaacauccuaca | cugggagguggaugu |
| 30b | uggcugggagguggauguuuacuuc (13) | cucagcu (14) | uuacuuc (15) |
| miR- | acucaaaaugggggcgcuuuccuuuuugucuguac | acucaaaaugggggc | gaagugcuucgauuu |
| 373 | ugggaagugcuucgauuuuggggugu (16) | gcuuucc (17) | uggggugu (18) |
| miR- | auguuggugcaaaaguaauuguggauuuugcuau | NA | aaaaguaauugugga |
| 548 | uacuuguauuuauuuguaaugcaaaacccgcaauu | uuuugcu (20) | |
| | aguuuugcaccaacc (19) | | |
| miR- | gcuccaguaacaucuuaaaguaaauaugcaccaaaa | uaaaguaaauaugca | NA |
| 559 | uuacuuuugguaaauacaguuuuggugcauauuu | ccaaaa (22) | |
| | acuuuaggauguuacuggagcuccca (21) | | |
| miR- | aaacacuugagcccagcgguuugaggcuacaguga | uuugaggcuacagug | ucucacuguagccuc |
| 1304 | gaugugauccugccacaucucacuguagccucgaa | agaugug (24) | gaacccc (25) |
| | ccccugggcucaagugauuca (23) | | |
| miR- | cucaggcugugacacucuagagggaagcgcuuucu | cucuagagggaagcg | aaagugcauccuuuu |
| 519 | guugucugaaagaaaggaaagugcauccuuuuaga | cuuucug (27) | agagugu (28) |
| | guguuacuguuugag (26) | | |
| miR- | agagguugcccuuggugaauucgcuuuauuuaug | agagguugcccuugg | aucacacaaaggcaac |
| 377 | uugaaucacacaaaggcaacuuuugu (29) | ugaauuc (30) | uuuugu (31) |
| miR- | cuccugacuccagguccuguguguuaccuagaaau | cuccugacuccaggu | acuggacuuggaguc |
| 378 | agcacuggacuuggagucagaaggc (32) | ccugugu (33) | agaaggc (34) |
| miR- | ccgggccccugugagcaucuuaccggacagugcug | caucuuaccggacag | uaacacugucuggua |
| 200 | gauuucccagcuugacucuaacacugucugguaac | ugcugga (36) | acgaugu (37) |
| | gauguucaaaggugacccgc (35) | | |
| miR- | cagcagcaauucauguuuugaaguguucuaaaugg | cagcagcaauucaug | caaaacgugaggcgc |
| 424 | uucaaaacgugaggcgcugcuaua (38) | uuuugaa (39) | ugcuaua (40) |
| miR- | cuagauaaguuauuaggugggugcaaagguaauu | aaagguaauugcagu | cgaaaacagcaauuac |
| 570 | gcaguuuuucccauuauuuuaauugcgaaaacagc | uuuuccc (42) | cuuugc (43) |
| | aauuaccuuugcaccaaccugauggagu (41) | | |

In some embodiments, the at least one miR, pre-miR or mimic is within an extracellular vesicle. In some embodiments, the at least one miR, pre-miR or mimic is within an exosome. In some embodiments, the at least one miR, pre-miR or mimic is within a liposome. In some embodiments, the at least one miR, pre-miR or mimic is encapsulated in a vesicle to improve deliverability, stability, half-life, targeting, or a combination thereof.

In some embodiments, over-expression comprises increasing expression of a naturally expressed miR. In some embodiments, over-expression comprises expression of an exogenous miR. In some embodiments, over-expression comprises expression of a pre-miR of a naturally expressed or exogenous miR. As used herein, an “exogenous miR”, refers to expression of a miR, pre-miR, miR mimic or other synthetic version of the miR that has been introduced into the cell. The cell may express an endogenous form of the miR, but this refers to an externally introduced synthetic form of the miR. As used herein, the terms “pre-miR” and “pri-miR” are used interchangeably and refer to a precursor RNA that is cleaved to produce a mature miR.

In some embodiments, the MSC further comprises at least one anti-cancer therapeutic agent. In some embodiments, the therapeutic agent is not one of the miRs of the invention. In some embodiments, the therapeutic agent is a chemotherapeutic. In some embodiments, the therapeutic agent is a radioactive agent. In some embodiments, the therapeutic agent is a ligand to a receptor on cancer cells. In some embodiments, the therapeutic agent is a membrane protein. In some embodiments, the therapeutic agent is TNF-related apoptosis-inducing ligand (TRAIL). In some embodiments, the therapeutic agent is membranal TRAIL (mTRAIL). In some embodiments, the mTRAIL is exogenous. In some embodiments, the therapeutic agent is Fas ligand (FasL). In some embodiments, the FasL is exogenous. In some embodiments, the MSCs comprise mTRAIL and FasL. In some embodiments, the extracellular vesicles also comprise, the at least one therapeutic agent. In some embodiments, the therapeutic agent is an anti-PD-L1 or anti-PD1 agent. In some embodiments, the therapeutic agent is a PD-L1/PD-1 blockade agent. In some embodiments, the therapeutic agent is a PD-L1/PD-1 therapeutic.

In some embodiments, the therapeutic agent is an oncolytic virus. In some embodiments, the MSCs further comprise an oncolytic virus. In some embodiments, the oncolytic virus is Newcastle disease virus (NDV). In some embodiments, the MSCs comprise at least one of mTRAIL, FasL and NDV. In some embodiments, the MSCs comprise a plurality of mTRAIL, FasL and NDV. In some embodiments, the MSCs comprise mTRAIL, FasL and NDV.

In some embodiments, the composition of the invention is for use in treating cancer in a subject in need thereof. In some embodiments, the composition of the invention is for use in decreasing PD-L1 expression in a target cell. In some embodiments, the composition of the invention is for use in decreasing PD-1 expression in a target cell. In some embodiments, the composition of the invention is for use in increasing immune surveillance in a subject in need thereof. In some embodiments, the composition of the invention is for use in decreasing escape from immune surveillance by a disease cell. In some embodiments, the disease cell is a cancer cell. In some embodiments, the composition of the invention is for use in decreasing proliferation of a cancer cell.

In some embodiments, the cancer is PD-L1 and/or PD-1 positive cancer. In some embodiments, the cancer is a PD-L1/PD-1 positive cancer and does not respond or is refractory to PD-L1/PD-1 based therapy. In some embodiments, the cancer is an exosomal PD-L1/PD-1 positive cancer. In some embodiments, reduction of exosomal PD-L1/PD-1 converts a refractory cancer to a responsive cancer. In some embodiments, the cancer is an exosomal PD-L1 positive cancer. In some embodiments, reduction of exosomal PD-L1 converts a refractory cancer to a responsive cancer. In some embodiments, the cancer is brain cancer. In some embodiments, the brain cancer is selected from glioma, and meningioma. In some embodiments, the brain cancer is selected from glioma, medulloblastoma and meningioma. In some embodiments, the brain cancer is selected from glioma, meningioma and a metastatic brain tumor. In some embodiments, the brain cancer is selected from glioma, pediatric astrocytoma, meningioma, medulloblastoma and a metastatic brain tumor. In some embodiments, the glioma is glioblastoma. In some embodiments, the glioblastoma is glioblastoma multiform. Other examples of brain cancers include, but are not limited to astrocytoma, craniopharyngioma, and oligodendroglioma. In some embodiments, the cancer is selected from brain cancer, lung cancer, breast cancer, melanoma, pancreatic cancer, and prostate cancer. In some embodiments, the cancer is selected from a brain cancer cell, a lung cancer cells, a breast cancer cell, a meningioma cell, a pancreatic cancer cell, a medulloblastoma cell, a glioma cell, a metastatic cell of a brain cancer and a prostate cancer cell. In some embodiments, the brain cancer has metastasized to the lung and/or breast. In some embodiments, the cancer cell is a cancer stem cell (CSC).

In some embodiments, the patient highly expresses PD-1. In some embodiments, an immune cell of the subject highly expresses PD-1. In some embodiments, the immune cell is a T cell. In some embodiments, the T cell is a cytotoxic T cell. In some embodiments, the immune cell is a natural killer cell (NK cell).

In some embodiments, the cell is an immune cell. In some embodiments, the cell is a cancer cell. In some embodiments, the cell is ex vivo. In some embodiments, the cell is in vivo. In some embodiments, the cell is in a subject. In some embodiments, the methods of the invention are performed ex vivo. In some embodiments, the contacting is ex vivo. In some embodiments, the expressing is ex vivo. In some embodiments, the MSCs are manipulated or modified ex vivo and then introduced into a subject.

In some embodiments, decreasing proliferation comprises decreasing self-renewal. In some embodiments, decreasing proliferation comprises increasing immune surveillance against the cell. In some embodiments, treating comprises decreasing self-renewal of a cancer cell. In some embodiments, treating comprises increasing immune surveillance against a cancer cell. In some embodiments, the increasing or decreasing comprises at least a 5%, 10%, 20%, 30%, 40%, 50%, 60%, 70%, 80%, 90%, 100%, 150%, 200%, 300%, 350%, 400%, 450% or 500% increase or decrease. Each possibility represents a separate embodiment of the invention.

In some embodiments, the MSC, extracellular vesicles from the MSC, or both comprise at least one exogenous microRNA (miR) that binds to and inhibits expression of PD-L1, PD-1 or both. In some embodiments, the MSC, extracellular vesicles or both comprises a plurality of such miRs. In some embodiments, the inhibiting is expression of PD-L1. In some embodiments, the inhibiting is expression of PD-1. In some embodiments, the inhibiting is expression of PD-L1 and PD-1. In some embodiments, the miR binds the 3′ untranslated region (UTR) of the target gene. In some embodiments, inhibiting expression comprises inhibiting translation. In some embodiments, inhibiting expression comprises degrading an mRNA. In some embodiments, inhibiting expression comprises reducing mRNA half-life.

In some embodiments, the miR is selected from the group consisting of miR-124, miR-29c, miR-383, miR-34 miR-30b, miR-373, miR-548, miR-559, miR-1304, miR-519, miR-377, miR-378, miR-200, miR-424, and miR-570. In some embodiments, the miR is selected from the group consisting of miR-124, miR-29c, miR-383, and miR-30b. In some embodiments, the miR is for inhibiting PD-L1 and the miR is selected from miR-124, miR-29c, miR-383, miR-373, miR-548, miR-559, miR-1304, miR-519, miR-377, miR-378, miR-200, miR-424, and miR-570. In some embodiments, the miR is for inhibiting PD-1 and the miR is selected from miR-124, miR-34 and miR-30B. In some embodiments, the miR is miR-124. In some embodiments, the at least one miR is at least two of miR-124, miR-29c and miR-383. In some embodiments, the at least one miR is at least two of miR-124, miR-34 and miR-30b. In some embodiments, the MSC, extracellular vesicles or both comprises exogenous miR-124, miR-29c and miR-383. In some embodiments, the MSC, extracellular vesicles or both comprises exogenous miR-124, miR-34 and miR-30b. In some embodiments, the MSC, extracellular vesicles or both over-express the at least one miR. In some embodiments, the at least one miR is not oncogenic. In some embodiments, the at least one miR does not increase self-renewal of a cancer cell. In some embodiments, the at least one miR is not miR-138 or miR-21. In some embodiments, only at least one of miR-124, miR-29c, miR-383, miR-34, miR-30b, miR-373, miR-548, miR-559, miR-1304, miR-519, miR-377, miR-378, miR-200, miR-424, and miR-570 are over-expressed, to the exclusion of any other miRs.

In some embodiments, the method of treating further comprises administering at least one anti-cancer treatment. In some embodiments, the anti-cancer treatment is an anticancer therapeutic agent. In some embodiments, the anti-cancer treatment is irradiating the subject. In some embodiments, the anti-cancer treatment is chemotherapy. In some embodiments the anti-cancer therapy is radiation therapy. In some embodiments, the anti-cancer therapy is PD-L1/PD-1 blockade.

In some embodiments, the MSC, extracellular vesicles, miRs, pre-miRs, mimics or combinations thereof sensitize the cancer to at least one anti-cancer therapy. In some embodiments, the MSC, extracellular vesicles, miRs, pre-miRs, mimics or combinations thereof sensitize the cancer to irradiation. In some embodiments, the MSC, extracellular vesicles, miRs, pre-miRs, mimics or combinations thereof sensitize the cancer to chemotherapy. In some embodiments, the MSC, extracellular vesicles, miRs, pre-miRs, mimics or combinations thereof sensitize the cancer to a PD-L1/PD-1 therapeutic. In some embodiments, the MSC, extracellular vesicles, miRs, pre-miRs, mimics or combinations thereof sensitize the cancer to irradiation and chemotherapy. In some embodiments, the MSC, extracellular vesicles, miRs, pre-miRs, mimics or combinations thereof sensitize the cancer to irradiation, chemotherapy and a PD-L1/PD-1 therapeutic. In some embodiments, the MSC, extracellular vesicles, miRs, pre-miRs, mimics or combinations thereof sensitize the cancer to at least one of irradiation, chemotherapy and a PD-L1/PD-1 therapeutic. In some embodiments, the MSC, extracellular vesicles, miRs, pre-miRs, mimics or combinations thereof protect a healthy cell form irradiation, chemotherapy or both. In some embodiments, the MSC, extracellular vesicles, miRs, pre-miRs, mimics or combinations thereof protect a healthy cell form irradiation. In some embodiments, the MSC, extracellular vesicles, miRs, pre-miRs, mimics or combinations thereof protect a healthy cell form chemotherapy. In some embodiments, a healthy cell is a non-cancerous cell.

In some embodiments, the composition further comprises a pharmaceutically acceptable carrier, excipient or adjuvant. As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide). In some embodiments, expression markers refer to RNA expression. In some embodiments, expression markers refer to protein expression.

Introduction of a gene, RNA, nucleic acid or protein into a live cell will be well known to one skilled in the art. As used herein, “introduction” refers to exogenous addition of a gene, miR or compound into a cell. It does not refer to increasing endogenous expression of a gene, protein or compound. Examples of such introduction include, but are not limited to transfection, lentiviral infection, nucleofection, or transduction. In some embodiments, the introduction is by transfection. In some embodiments, the introduction is by lentiviral infection. In some embodiments, the introducing occurs ex vivo. In some embodiments, the introducing occurs in vivo. In some embodiments, the introducing occurs in vivo or ex vivo. In some embodiments, the introduction comprises introducing a vector comprising the gene of interest. In some embodiments, a miR, pre-miR or vector comprising the miR or pre-miR are introduced into the MSC. In some embodiments, the pre-miR is introduced. In some embodiments, the miR is introduced. In some embodiments, a vector comprising the miR, wherein the miR is configured for expression in the MSC is introduced.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like. In some embodiments, the vector, miR, lncRNA or RNA inhibitory molecule are transfected into the MSC.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

In some embodiments, introduction of a gene of interest comprises introduction of an inducible vector, wherein administration of a drug to the cell will induce expression of the gene of interest. Drug inducible vectors are well known in the art, some non-limiting examples include tamoxifen-inducible, tetracycline-inducible and doxycycline-inducible. In some embodiments, the inducible-vector is introduced to the MSC ex-vivo and the MSC is contacted with the inducing drug in-vivo. In this way expression of the induced gene, and as a result priming or differentiation of the MSC, only occurs in-vivo. In some embodiments, priming or differentiation of the MSC only occurs after the MSC has homed to a location in the body of a subject.

In some embodiments, introducing comprises introducing a modified RNA. The term “modified RNA” refers to a stable RNA that maybe introduced into the cytoplasm of the cell and will there be translated to protein. Such an RNA does not require transcription for protein expression and thus will more quickly produce protein and is subject to less regulation. Modified RNAs are well known in the art.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intratumor, intramuscular, or intraperitoneal.

In some embodiments, a method of the invention comprises administration of a combination of cells and optionally their exosomes. In some embodiments, unmodified and modified MSC are administered together. In some embodiments, unmodified MSCs are administer. In some embodiments, modified MSCs are administered. In some embodiments, exosomes from any of these cell types are also administered together. In some embodiments, the just the exosomes are administered.

The term “extracellular vesicles”, as used herein, refers to all cell-derived vesicles secreted from a cell including but not limited to exosomes and microvesicles. “Exosome”, as used herein, refers to cell-derived vesicles of endocytic origin, with a size of at least 30-120 nm. In some embodiments, the exosomes are secreted from a prokaryotic cell. In some embodiments, the exosomes are secreted from a eukaryotic cell. In some embodiments, the exosomes are secreted from a plant cell. In some embodiments, the exosomes are secreted from a mammalian cell. In some embodiments, the exosomes are secreted from a human cell. In some embodiments, the exosomes are secreted from a stem cell. In some embodiments, exosomes are secreted from MSCs. As a non-limiting embodiment, for the generation of exosomes cells are maintained with Opti-MEM, MEM or DMEM and human serum albumin or 5% FBS that was depleted from exosomes. In some embodiments, exosomes comprise all extracellular vesicles. In some embodiments, exosomes are isolated from milk.

“Microvesicles”, as used herein, refers to cell-derived vesicles originating from the plasma membrane, with a size of 100-1000 nm, and secreted from MSCs.

Exosomes, extracellular vesicles, or microvesicles can be obtained by growing MSCs in culture medium with serum depleted from exosomes or in serum-free media such as OptiMeM and subsequently isolating the exosomes by ultracentrifugation. Other methods associated with beads, columns, filters and antibodies are also employed. In some embodiments, the cells are grown in hypoxic conditions or incubated in medium with low pH so as to increase the yield of the exosomes. In other embodiments, the cells are exposed to radiation so as to increases exosome secretion and yield. In some embodiments, the exosomes are suspended in appropriate carrier for administration.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Example 1

### CH- and UC-MSCs Decrease PD-L1 Expression in Cancer Cells

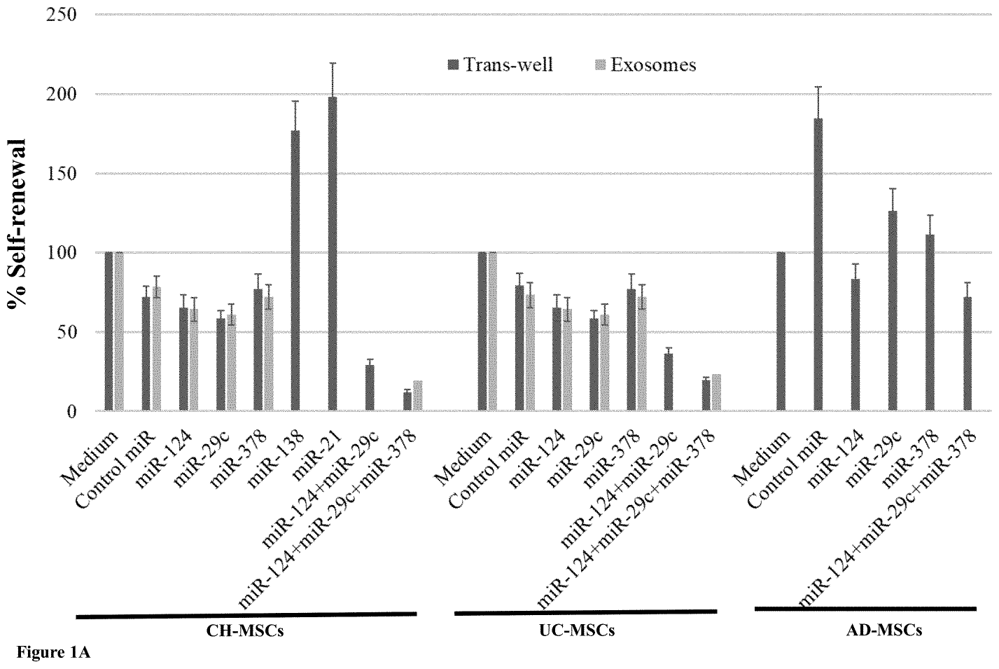

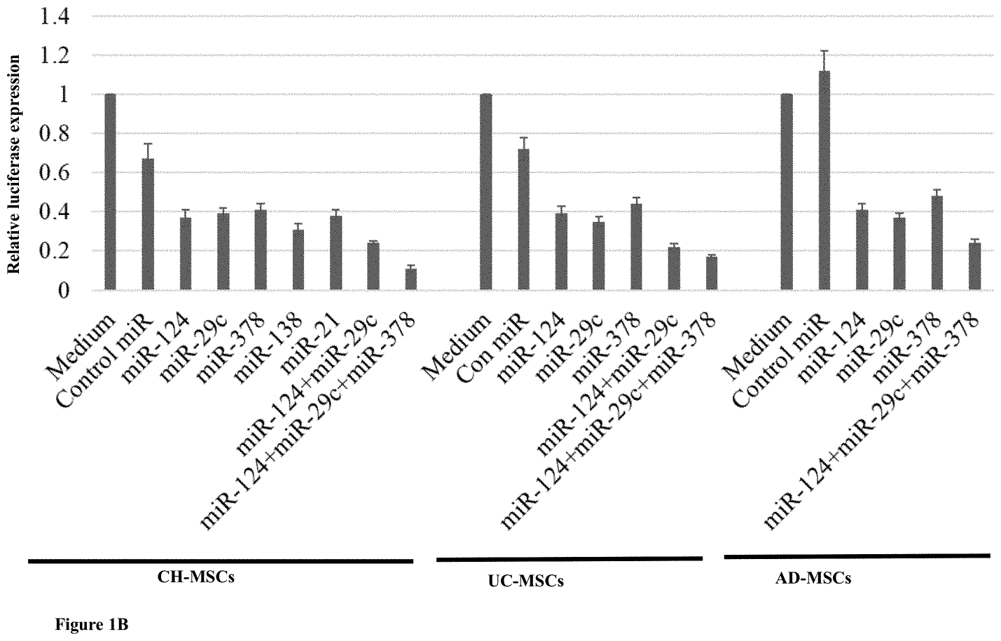

Certain MSCs have previously been shown to exert antitumor effects on a wide variety of cancer cells. It was unexpectedly discovered that MSCs from certain sources also downregulated PD-L1 expression in cancer cells. MSCs derived from chorionic placenta (CH), umbilical cord (UC) and adipose tissue (AD) were tested for their ability to inhibit self-renewal in glioma stem cells (GSCs) and to decrease expression of PD-L1. GSCs were transduced with a lentiviral expression vector containing a luciferase reporter construct in which the 3′ UTR of PD-L1 was inserted after the luciferase open reading frame. These GSCs were grown in a trans-well dish with either CH-MSCs, UC-MSCs or AD-MSCs and self-renewal (FIG. 1A) as well as luciferase were measured. (FIG. 1B). Luciferase activity was measured by a standard luciferase assay using the Promega luciferase assay kit according to the manufacturer's protocol. Cells were lysed 48 hours after coculture was started and renilla luciferase was used for normalization. Self-renewal was measured by examining GSC's ability to form secondary neurospheres. Briefly, after coculture, GSCs were plated in a 24-well dish at a density of 100 cells/well through limiting dilution. The number of spheroids that formed after 14 days was measured. Only spheroids with at least 20 cells were scored. The number of spheroids formed from cells cultured without MSC was used as the baseline (100% self-renewal) and results are given as a percentage of this baseline. Both CH- and UC-MSCs decreased self-renewal of the GSCs by over 20%, whereas AD-MSC actually had a strong pro-tumor effect with a greater than 80% increase in self-renewal. CH-MSCs also decreased luciferase activity by a third and similar results were observed for UC-MSCs. AD-MSCs showed no such effect on the PD-L1 3′UTR (FIG. 1B).

These MSC effects were seen in transwell culture, when the MSCs and GSCs were not physically in contact, thus it was hypothesized that extracellular vesicles from the MSCs mediate the decrease in self-renewal. To test this, exosomes from CH- and UC-MSCs were isolated according to standard protocol and added to GSCs in culture. Exosomes alone showed a comparable effect to that seen in the transwell culture experiments (FIG. 1A, 3B).

### Example 2

### miR-124, miR-29c and miR-378 Regulate PD-L1 Expression and Decrease Self-Renewal

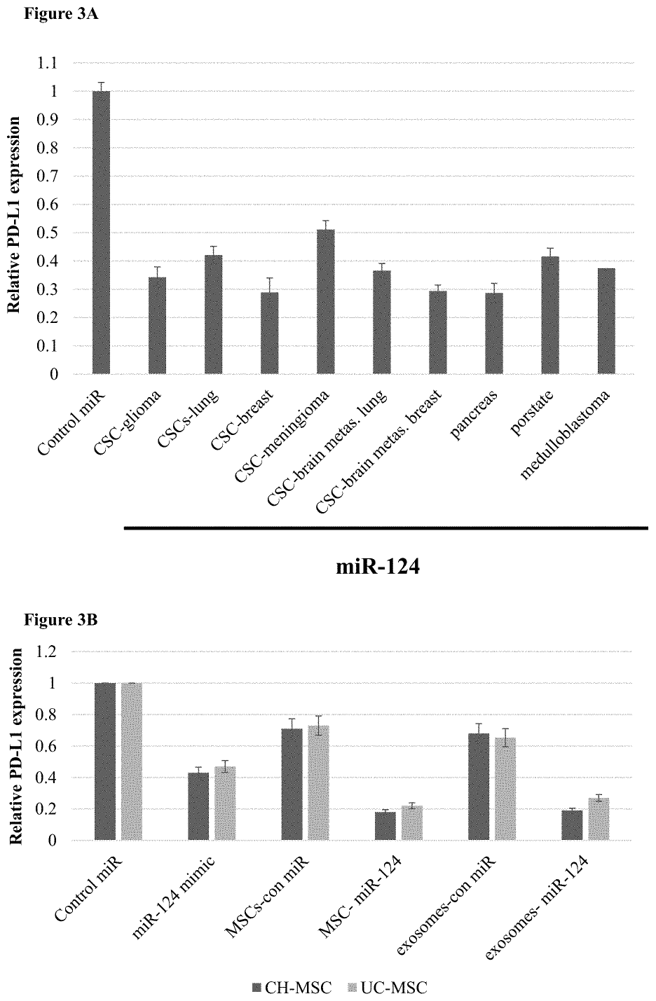

Next, several microRNAs (miRs) were tested in concert with the MSCs. CH-, AD- and UC-MSCs were transfected with vectors containing pre-miR for miR-124, miR-29c, miR-378, miR-138 and miR-21 or combinations of these miRs, cocultured in trans-well plates with GSCs and the effect on GSC self-renewal and luciferase expression was monitored. The five miRs all decreased luciferase expression in all MSCs tested, indicating that all five miRs directly regulate PD-L1 expression (FIG. 1B). This effect was stronger than the effect observed with MSCs alone and was also observed when exosomes from the MSCs were added directly (FIG. 3B).

Interestingly the effect on self-renewal of GSCs was not the same for all 5 miRs. miR-138 and miR-21 had very strong pro-self-renewal effects, nearly doubling the GSC's self-renewal (FIG. 1A). Thus, though these two miRs decrease PD-L 1 expression, they are poor candidates for cancer treatment. In contrast, each of miR-124, miR-29c, and miR-378 had a negligible effect on self-renewal when expressed in CH- and UC-MSCs, though a more pronounced effect was observed when expressed in AD-MSCs. miR-124 in particular brought the percent self-renewal below untreated levels (FIG. 1A). Though each miR alone did not have a strong effect on self-renewal when expressed in CH- and UC-MSCs, the combined expression of miR-124 and miR-29 improved the effect of the MSCs by greater than 2.5-fold (27.9% vs 70.8% reduction with CH, 20.7% vs. 63.8% reduction with UC). Expression of miR-124, miR-29c, and miR-378 resulted in an 87.9% reduction after CH co-culture, and an 85.8% reduction after UC coculture. Exosomes from these cells had a similar effect (FIG. 1A). As, these 3 miRs had a positive effect on cancer stem cell self-renewal, there combined effect on PD-L1 expression was also examined. CH-MSCs expressing miR-124, miR-29c, and miR-378 showed a strong synergistic effect, decreasing luciferase reporter expression by ˜90% (FIG. 1B).

### Example 3

### miR-124, miR-34 and miR-30b regulate PD-1 expression and decrease self-renewal

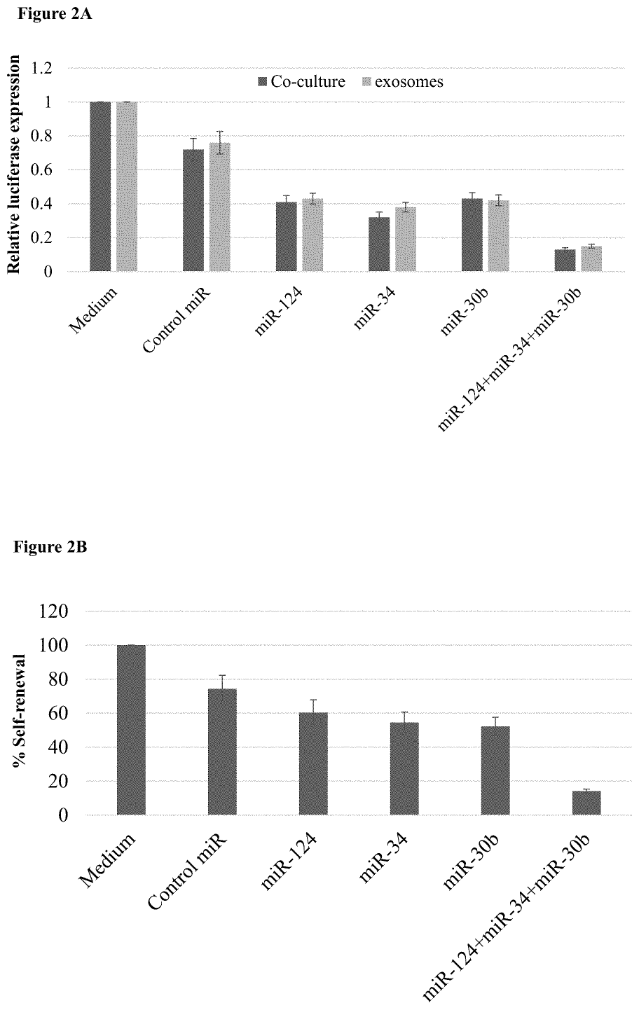

PD-L1 is only half of the immune surveillance axis, its receptor PD-1 is expressed on immune cells and is required for cancer cell immune inhibition. Unexpectedly CH-MSCs and their exosomes also decreased PD-1 expression in a similar reporter assay experiment in which luciferase with the PD-1 3′ UTR was expressed in HEK293 cells (FIG. 2A). Thus, CH-MSC act on both PD-L1 and PD-1 to decrease their expression.

CH-MSCs were also transfected with miR-124, miR-34, miR-30b and a combination of all three and then PD-1 expression was assessed. All three miRs enhanced the PD-1 reduction, and the MSCs expressing all three together decreased PD-1 expression by ˜90% (FIG. 2A). The effect was the same whether cells were cocultured, or whether exosomes were added. miR-34 has already been reported to target PD-L1, thus this miR and miR-124 are both effective at targeting both sides of the PD-L1/PD-1 axis.

It was necessary to check if these miRs might have an adverse effect on cancer cell self-renewal as was observed for miR-138 and miR-21. Surprisingly, not only did each miR individually enhance the anti-self-renewal effect of the CH-MSCs, but MSCs expressing all three miRs brought about a greater than 85% reduction in self-renewal in the GSCs (FIG. 2B).

### Example 4

### miR-124 Reduces PD-L1 Expression in a Wide Variety of Cancers

As miR-124 had such a strong beneficial effect on the PD-L1/PD-1 axis and also on cancer stem cell (CSC) self-renewal and has previously be shown to sensitive cancer stem cells to radiation while protecting normal cell from radiation-induced injury, its effectiveness on a wide variety of cancers was investigated. Cell lines derived from glioma stem cells, lung cancer stem cells, breast cancer stem cells, meningioma stem cells, stem cells from brain cancer than metastasized to the lung, stem cells from brain cancer than metastasized to the breast, pancreas cancer cells, prostate cancer cells and medulloblastoma cells were all incubated with exosomes from CH-MSCs that expressed either a control miR or miR-124 and surface expression of PD-L1 was measured by FACS (FIG. 3A). Expression of PD-L1 in cells contacted with a control miR was used as a control and set as 1 so as to allow comparisons across cell types. In all samples tested miR-124 produced a robust decrease in PD-L1 expression of at least 50%.

The effectiveness of free miR-124 was next checked against the effect of miR-124 expressed in CH- or UC-MSCs. Free miR-124 on its own more effectively lowered PD-L1 expression than did MSCs on their own, or their exosomes, however, as expected, MSCs expressing miR-124, or their exosomes, were even more effective (FIG. 3B).

### Example 5

### Addition of Other Anticancer Agents to the MSCs Improves their Effectiveness

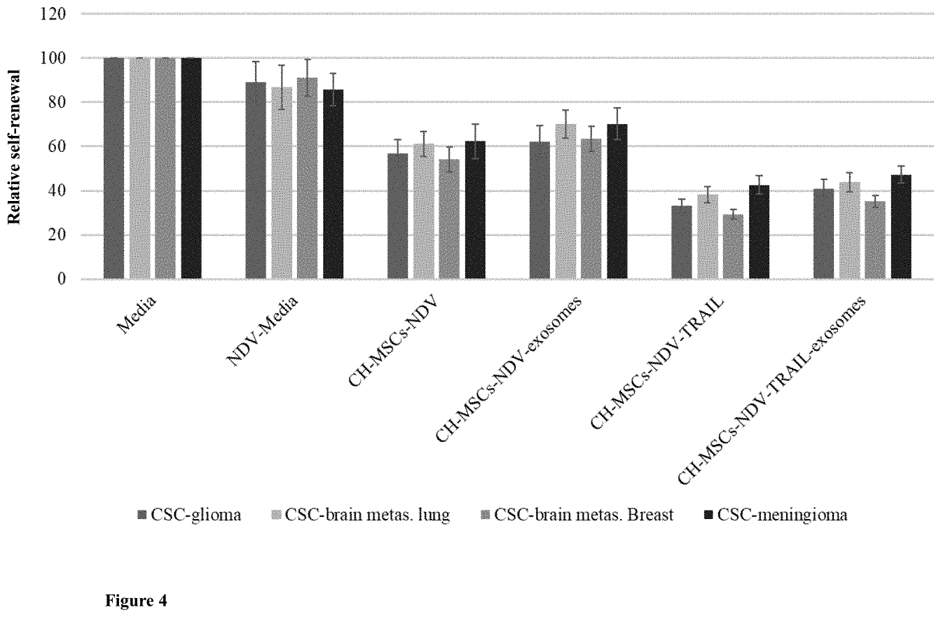

Several other anti-cancer agents can also be added to MSCs to improve their overall effectiveness. Four different brain cancer stem cells, glioma stem cells, meningioma stem cells, brain cancer stem cells that metastasized to the lungs, and that metastasized to the breast were all grown in a transwell co-coculture with various cytotoxic agents and combinations of agents and MSCs and self-renewal was measured as described hereinabove. Plain media was added as control, and the following treatments were tested: media containing the oncolytic New Castle Virus (NDV), CH-MSCs previously infected with NDV, exosomes from those infected MSCs, CH-MSCs previously infected with NDV and expressing membranal TRAIL (mTRAIL) and exosomes from those MSCs (FIG. 4). Fas ligand (FasL) expression is also tested as a possible anti-cancer agent to add to the cells. MSCs were infected with 1 multiplicity of infection (MOI) of NDV (MTH-68) for 2 hours, after which the cells were washed three times and incubated in fresh medium before transfer to transwell co-culture. mTRAIL and FasL expression is achieved via lentiviral infection with a mTRAIL or FasL expression plasmid.

The NDV on its own only decreased self-renewal by 10-15%, whereas MSCs expressing the virus and their exosomes had a much stronger (30-55%) effect. The triple combination of the MSCs, the virus and mTRAIL was even better, with the cells themselves decreasing self-renewal by 58-70% and the exomes from those cells producing a 53-65% reduction (FIG. 4).

### Example 6

### Additional miRs Regulate PD-L1 Expression.

Several additional miRs were tested for their ability to regulate PD-L1 expression using exosomes and trans-well experiments. The tested miRs were mir-105, miR-373, miR-548, miR-559, miR-1304, miR-522, miR-519, miR-377, miR-378, miR-200, miR-424, miR-570, and miR-34.

Exosomes from CH-MSCs were transfected with a control miR or one of the tested miRs. Thereafter, exosomes were collected and co-cultured with GSCs containing a luciferase reporter construct. After 24 hours of incubation luciferase expression was measured. Even though exosomes from untransfected or control miR transfected CH-MSCs lowered the luciferase expression (see Example 1), this level of luciferase activity was set to 1.

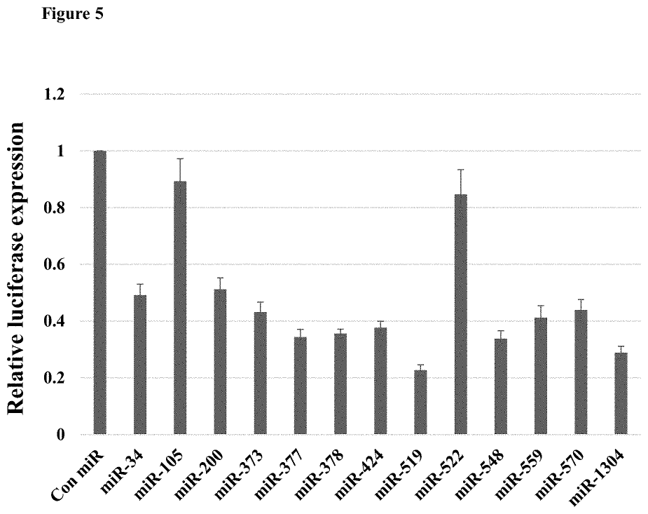

11 of the 13 tested miRs exhibited reduced luciferase expression as compared to the control miR (FIG. 5). Two the miRs, miR-105 and miR-522, though predicted computationally to inhibit PD-L1 did not in fact reduce luciferase expression. Exosomes from UC-MSCs showed similar results, with the same 11 miRs reducing luciferase expressiong as compared to the control miR. GSCs were also grown in a trans-well dish with the transfected MSCs themselves. Transwell culture showed a comparable effect to that seen in the exosomes experiment. The tested miRs were thus found to inhibit both expression of PD-L1 and secretion of exosomal PD-L1.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of decreasing Programmed Death Ligand 1 (PD-L1) expression, PD-1 expression, or both, in a cell, the method comprising contacting said cell with a chorionic placenta (CH) mesenchymal stem cell (MSC), extracellular vesicle from said CH-MSC or both, optionally wherein said cell is a cancer cell.

2. (canceled)

3. (canceled)

4. (canceled)

5. A method of decreasing proliferation of a cancer cell, the method comprising contacting said cancer cell with a chorionic placenta (CH)MSC, extracellular vesicle from said MSC or both.

6. The method of claim 1, wherein the cell is in a subject and said method comprises administering to said subject said CH-MSC, extracellular vesicle from said CH-MSC or both.

7. The method of claim 5 or 6, wherein said decreasing comprises:
a. decreasing self-renewal of said cell and increasing immune surveillance against said cell, or
b. at least a 20% decrease.

8. (canceled)

9. The method of claim 1, wherein said MSC, extracellular vesicle from said MSC or both, comprise at least one exogenous microRNA (miR) that binds to and inhibits expression of PD-L1, PD-1 or both, optionally wherein said at least one exogenous miR is not oncogenic, binds to a 3′ untranslated region (UTR) of PD-L1 or both.

10. (canceled)

11. (canceled)

12. The method of claim 9, wherein said at least one exogenous miR is selected from the group consisting of: miR-124, miR-29c, miR-383, miR-373, miR-548, miR-559, miR-1304, miR-519, miR-377, miR-378, miR-200, miR-424, miR-570, and miR-34 or the group consisting of: miR-373, miR-548, miR-559, miR-1304, miR-519, miR-377, miR-378, miR-200, miR-424, miR-570, and miR-34.

13. (canceled)

14. (canceled)

15. The method of claim 9, wherein said at least one exogenous miR binds to a 3′ UTR of PD-1 and is selected from the group consisting of: miR-124, miR-34 and miR-30b.

16. (canceled)

17. (canceled)

18. (canceled)

19. The method of claim 9, wherein said CH-MSC, exosomes from said CH-MSC, or both comprise miR-124 and
a. and at least one miR selected from the group consisting of miR-29c, miR-383, miR-34, miR-30b, miR-373, miR-548, miR-559, miR-1304, miR-519, miR-377, miR-378, miR-200, miR-424, and miR-570,
b. and at least one miR selected from the group consisting of miR-29c, miR-383, miR-34, and miR-30b;
c. miR-29c and miR-383; or
d. miR-34 and miR-30b.

20. The method of claim 1, wherein said cell is a cancer cell and wherein said cancer cell is selected from a brain cancer cell, a lung cancer cell, a breast cancer cell, a melanoma cell, a meningioma cell, a pancreatic cancer cell, a prostate cancer cell, a medulloblastoma cell, a glioma cell and a metastatic cell of a brain cancer optionally wherein brain cancer cell is a glioblastoma multiform (GBM) cell or a GBM stem cell.

21. (canceled)

22. (canceled)

23. (canceled)

24. The method of claim 1, wherein said CH-MSC or exosomes expresses at least one at least one anti-cancer therapeutic agent selected from exogenous membranal TRAIL (mTRAIL) protein or Fas ligand (FasL) and an oncolytic virus, optionally wherein said oncolytic virus is Newcastle disease virus (NDV).

25. (canceled)

26. (canceled)

27. A pharmaceutical composition comprising at least one of:
a. a chorionic placenta (CH) MSC expressing at least one exogenous miR selected from miR-124, miR-29c miR-383 miR-373, miR-548, miR-559, miR-1304, miR-519, miR-377, miR-378, miR-200, miR-424, miR-570, and miR-34;
b. extracellular vesicles derived from (a);
c. a combination thereof.

28. (canceled)

29. (canceled)

30. The composition of claim 27, wherein said MSC further comprises at least one anti-cancer therapeutic agent.

31. The composition of claim 30, wherein said at least one anti-cancer therapeutic agent is selected from mTRAIL protein or (FasL) an oncolytic virus, and at least one exogenous miR selected from miR-34 and miR-30b, optionally wherein said oncolytic virus is NDV.

32. (canceled)

33. The composition of any one of claim 27, comprising at least two miRs selected from miR-124, miR-29c and miR-383.

34. The composition of claim 27, comprising miR-124, miR-29c and miR-383.

35. (canceled)

36. (canceled)

37. (canceled)

38. (canceled)

39. (canceled)

40. (canceled)

41. A method of treating a Programmed Death Ligand 1 (PD-L1) positive cancer in a subject in need thereof, the method comprising administering to the subject a pharmaceutical composition of claim 27.

42. The method of claim 41, wherein said treating comprises at least one of
a. decreasing PD-L1 expression by said PD-L1 positive cancer;
b. decreasing proliferation of said PD-L1 positive cancer;
c. decreasing PD-1 expression by said subject and
d. converting a cancer refractory to PD-L1/PD-1 based therapy to a cancer responsive to PD-L1/PD-1 based therapy.

43. (canceled)

44. (canceled)

45. (canceled)

46. The method of claim 41, to wherein
a. said MSC is allogenic or autologous to said subject
b. said PD-L1 positive cancer is selected from a brain cancer cell, a lung cancer cell, a breast cancer cell, a melanoma cell, a meningioma cell, a pancreatic cancer cell, a prostate cancer cell, a medulloblastoma cell, a glioma cell and a metastatic cell of a brain cancer; or
c. said PD-L1 positive cancer is a brain cancer, optionally wherein said brain cancer is glioblastoma multiform (GBM).

47. (canceled)

48. (canceled)

49. (canceled)

50. (canceled)

51. (canceled)

52. (canceled)

53. (canceled)

54. The method of claim 41, further comprising administering at least one anticancer treatment, optionally wherein said at least one anticancer treatment is a PD-L1/PD-1 therapeutic.

55. (canceled)

56. The method of claim 54, wherein said at least one anticancer treatment is selected from irradiating said subject and chemotherapy and wherein said MSCs, exosomes, miRs, or a combination thereof
a. sensitize said PD-L1 positive cancer to irradiation, chemotherapy or both, or
b. protect healthy cells from said irradiation, chemotherapy or both, or
c. both.

57. (canceled)

58. (canceled)
